(19) United States
(12) Patent Application Publication
(10) Pub. No.: US 2002/0148471 A1
(43) Pub. Date: Oct. 17, 2002

(54) Radiative artificial respiration system with carbon dioxide absorbent and canister for use in same

(76) Inventor: Go Hirabayashi, Tokyo (JP)

Correspondence Address: FLEIT, JACOBSON, COHN, PRICE,, HOLMAN & STERN, 400 Seventh Street, N.W., Washington, DC, 20004, US

(21) Appl. No.: 10/092,904

(22) Filed: Mar. 8, 2002

(30) Foreign Application Priority Data
Mar. 9, 2001 (JP) 2001-067596
Feb. 22, 2002 (JP) 2002-047109

(51) Int. Cl. A61M 15/00; A61M 16/10; F24F 5/00; A62B 7/00; A61M 16/00; A62B 7/10; A62B 19/00; A62B 23/02

(52) U.S. Cl. 128/205.12; 128/203.12; 128/204.16

(57) ABSTRACT

The present invention allows a low-flow anesthesia method to be executed while hindering temperature increases in a carbon dioxide absorbent, thereby reducing evaporation of water from the carbon dioxide absorbent and the amount of condensation formed in an anesthetic circuit, but without increasing the amount of decomposed compounds.

With a closed circulatory artificial respiration system, when a low-flow anesthesia method is to be carried out in which the total amount of the fresh air containing oxygen and a volatile anesthetic is 2 l/min. or less, a carbon dioxide absorbent, which generates heat when absorbing carbon dioxide, is allowed to efficiently radiate heat to minimize increases in the temperature of the carbon dioxide absorbent, thus reducing evaporation of water from the carbon dioxide absorbent and the amount of condensation formed in an anesthetic circuit, but without increasing the amount of decomposed compounds produced by the reaction between the volatile anesthetic and the carbon dioxide absorbent.

### FIELD OF THE INVENTION

The present invention relates to a closed circulatory artificial respiration system that repeats a process of using a carbon dioxide absorbent to absorb carbon dioxide contained in breath discharged from a patient in order to remove the carbon dioxide, then supplying fresh gas into the circuit in order to allow it to combine with recycled gas, and subsequently allowing the patient to absorb the gas as an inspiration, as well as a canister for use in this system.

### DESCRIPTION OF PRIOR ART

Artificial respiration systems can be classified into three types: those having an open (non-rebreathing) circuit, those having a semi-closed circulatory (partial rebreathing) circuit, and those having a closed circulatory (rebreathing) circuit. Open circuit systems exhaust all of the oxygen, carbon dioxide, and anesthetic gas discharged from a patient. Closed circulatory circuit systems remove carbon dioxide and recycle almost all the oxygen and anesthetic gas discharged from the patient. Semi-closed circulatory circuit systems are the intermediary between closed circulatory and open circuit systems. They remove carbon dioxide and recycle oxygen and anesthetic gas, but always supply a certain amount of oxygen and anesthetic gas, while exhausting a certain amount of oxygen and anesthetic gas. The recycle rate is determined by fresh gas flow. As the fresh gas flow decreases, the recycle rate of anesthetic gas increases, whereas the amount of exhausted gas decreases. In a broad sense, semi-closed circulatory systems are a subset of the closed circulatory systems. Most artificial respirators used for anesthesia are of the closed circulatory type.

A low-flow anesthesia method refers to closed circulatory anesthesia in which a fresh gas containing oxygen and anesthetic gas has a flow amount of 2 l/min. or less. A high-flow anesthesia method refers to closed circulatory anesthesia in which fresh gas containing oxygen and anesthetic gas have a flow amount of more than 2 l/min. (in a narrow sense, a flow amount of 1 to 2 l/min. is sometimes referred to as an “intermediate flow”). Low-flow anesthesia is more advantageous than high-flow anesthesia in terms of cost and environmental impact because the anesthetic gas is efficiently recycled, a reduced amount of oxygen and anesthetic gas is consumed, and a reduced amount of gas is exhausted. Low-flow anesthesia may result in the formation of a large amount of condensation. This is presumably because the canister insufficiently radiates heat and the carbon dioxide absorbent insufficiently transmits heat, so that the local temperature of the carbon dioxide absorbent increases significantly, thus causing more water to be evaporated from the absorbent.

Further, it has been pointed out that low-flow anesthesia may disadvantageously lead to an increase in the concentration of toxic decomposed compounds (compound A and carbon monoxide) in the circuit, which compounds are produced by the reaction between a volatile anesthetic and the carbon dioxide absorbent. For example, low-flow anesthesia using sevoflurane (the trademark of a volatile anesthetic manufactured by Maruishi Pharmaceutical Co., Ltd.) may result in an increase in the concentration of compound A, a decomposed compound, in the circuit. While it has not been confirmed that compound A has renal toxicity to human beings, its renal toxicity to rats has been experimentally determined. A good deal of controversy has arisen about the use of sevoflurane at a low flow rate. Further, it has been reported that low-flow anesthesia using forane (the trademark of a volatile anesthetic manufactured by Dinapot Co., Ltd.) leads to an increase in the concentration of carbon monoxide in the circuit. An increase in the local temperature of the carbon dioxide absorbent increases the production of decomposed compounds, while radiation of heat from the carbon dioxide absorbent reduces the production of decomposed compounds.

### SUMMARY OF THE INVENTION

It is an object of the present invention to carry out a low-flow anesthesia method while minimizing increases in the temperature of the carbon dioxide absorbent, thus reducing evaporation of moisture from the carbon dioxide absorbent and the amount of condensation formed in the anesthetic circuit, but without increasing the amount of decomposed compounds produced by the reaction between a volatile anesthetic and the carbon dioxide absorbent. Another object of the present invention is to develop a new artificial respirator that can inexpensively and easily solve the above problems with low-flow anesthesia. This will serve as the basis for the diffusion of low-flow anesthesia, which reduces costs, prevents operation-room employees from being exposed to gas, and reduces the amount of anesthetic gas exhausted to the atmosphere.

These and other objects are attained by a closed circulatory artificial respiration system according to the present invention which repeats a process of using a carbon dioxide absorbent to absorb carbon dioxide contained in breath discharged from a patient in order to remove the carbon dioxide, then supplying fresh gas containing oxygen and an anesthetic into the circuit in order to allow it to combine with recycled gas, and subsequently allowing the patient to absorb the fresh (mixed) gas as an inspiration. Hence, when a low-flow anesthesia method is to be carried out in which the total amount of the fresh air containing the oxygen and volatile anesthetic is 2 l/min. or less, the carbon dioxide absorbent, which generates heat when absorbing carbon dioxide, is allowed to efficiently radiate heat to minimize the increase in temperature of the carbon dioxide absorbent, thus reducing evaporation of moisture from the carbon dioxide absorbent and the amount of condensation formed in the anesthetic circuit, but without increasing the amount of decomposed compounds produced by the reaction between the volatile anesthetic and the carbon dioxide absorbent.

Further, to execute this low-flow anesthesia method, it is preferable to form a case of a canister which is composed of aluminum or copper (or some other material having a heat conductivity equivalent to that of aluminum or copper) to accommodate the carbon dioxide absorbent therein in order to enhance heat radiation, and/or to use a canister having radiator panels arranged therein and formed of the same type of material as mentioned above. However, material with a high heat conductivity such as aluminum may be corroded by the carbon dioxide absorbent, so it is effective to coat this material, for example, by plating the material with chromium. To allow the carbon dioxide absorbent to efficiently radiate heat, it is effective to add, for example, a Peltier cooling method using a Peltier effect. Further, apparatuses that are not sufficiently safe (e.g., apparatuses that are prone to break down) are not desirable as medical instruments. The addition of the Peltier cooling method allows the temperature of the carbon dioxide absorbent to be electrically controlled, but may cause failures and increase the product price. Hence, in view of the product price and in the interest of simplicity, the Peltier cooling method is not the only measure to take. In fact, it may be sufficiently effective to execute an air cooling method that externally ventilates the canister to facilitate radiation, or to use only the above-mentioned radiative canister without the addition of the Peltier cooling method or the air cooling method. Such a method also constitutes an inexpensive and simple carbon dioxide absorbent radiator.

To allow the technical significance of the present invention to be easily understood, the inventors' experiments will be described in which closed circulatory anesthetic circuits were each equipped with a Peltier cooling radiator with a carbon dioxide absorbent or a conventional plastic canister, and pigs were put under low-flow anesthesia with sevoflurane (fresh gas flow of 0.6 l/min.) for 12 hours. A group of Peltier cooling radiators with carbon dioxide absorbents and a group of conventional plastic canisters were all installed in the respective closed circulatory anesthesia circuits (Fabius: a trademark of Dräger, Lübeck, Germany), and 1.5 liters of fresh soda lime (Drägersorb 800 plus: a trademark of Dräger, Lübeck, Germany) was used as a carbon dioxide absorbent. In the following description, the results are shown in terms of means±S.D., statistical data is based on Mann-Whitney U tests, and a value p<0.05 is considered to be significant.

1) Variation in the Temperature of the Carbon Dioxide Absorbent

In the group of conventional plastic canister apparatuses, the carbon dioxide absorbents reached their maximum temperature of 40.6±1.6° C. four hours after the start of the experiments. In contrast, in the group of Peltier cooling radiators with carbon dioxide absorbents, the temperature of the carbon dioxide absorbents reached 29.5±0.5° C. one hour after the start of the experiments and then remained at about 30° C. until the experiments were completed.

2) Water Content of the Carbon Dioxide Absorbent

Before the start of the experiments, the water content of the carbon dioxide absorbent was 15.5±0.1% in the group of plastic canister apparatuses and 15.4±0.1% in the group of Peltier cooling radiators with carbon dioxide absorbents. Thus, in this respect, there was no statistical difference between these groups. After the completion of the experiments, the water content of the carbon dioxide absorbent decreased down to 2.5±0.4% in the group of plastic canister apparatuses and to 11.9±1.5% in the group of Peltier cooling radiators with carbon dioxide absorbents, indicating that the carbon dioxide absorbents dried in both groups. However, the water content decreased more significantly in the group of plastic canister apparatuses than in the group of Peltier cooling radiators with carbon dioxide absorbents (p<0.001). The amount of water lost from the carbon dioxide absorbent was 69.5±22.0 g in group of plastic canister apparatuses and 0.7±7.8 g in the group of Peltier cooling radiators with carbon dioxide absorbents. That is, in the latter group, practically no water was lost (p<0.001).

3) The Transition of the Condensation Formation in the Expiratory Valve and Inspiratory Valve

In the group of plastic canister apparatuses, no condensation is formed in the expiratory valve, whereas a large amount is formed in the inspiratory valve. On the other hand, in the group of Peltier cooling radiators with carbon dioxide absorbents, a small amount of condensation is formed in the expiratory valve, whereas none is formed in the inspiratory valve.

4) Transition of the Concentration of Compound A in the Circuit

At the start of low-flow anesthesia, the concentration of compound A in the circuit was 20.3±3.3 ppm in the group of plastic canister apparatuses and 18.0±2.1 ppm in the group of Peltier cooling radiators with carbon dioxide absorbents. Thus, in this respect, there was no significant difference between these groups. One hour after the start of low-flow anesthesia, the group of plastic canister apparatuses reached a maximum concentration of 26.6±2.1 ppm, whereas the group of Peltier cooling radiators with carbon dioxide absorbents reached a maximum concentration of 18.1±2.2 ppm. Comparison of these values indicates that the group of Peltier cooling radiators with carbon dioxide absorbents had a lower concentration than the group of plastic canister apparatuses and the difference between these groups was significant (p<0.01). Furthermore, the total exposure amount was 274.8±11.2 ppm·hr in the group of plastic canister apparatuses and 193.8±17.4 ppm ·hr in the group of Peltier cooling radiators with carbon dioxide absorbents according to the present invention. Thus, the group of Peltier cooling radiators with carbon dioxide absorbents served to significantly reduce the concentration of compound A in the circuit (p<0.001).

These results support the following claims: with a closed circulatory artificial respiration system that repeats a process of using a carbon dioxide absorbent to absorb carbon dioxide contained in expiration discharged from a patient in order to remove the carbon dioxide, then supplying fresh gas containing oxygen and anesthetic into the circuit in order to allow it combine with regulated gas, and subsequently allowing the patient to absorb fresh(mixed) gas as an inspiration, when a low-flow anesthesia method is to be carried out, in which the total amount of fresh gas containing the oxygen and volatile anesthetic is 2 l/min. or less, the carbon dioxide absorbent, which generates heat when absorbing carbon dioxide, can efficiently radiate heat to minimize temperature increases of the carbon dioxide absorbent by attaching the present invention. As a result, evaporation of moisture from the carbon dioxide absorbent is reduced, as is the amount of condensation formed in an anesthetic circuit, but without increasing the amount of decomposed compounds produced by the reaction between the volatile anesthetic and the carbon dioxide absorbent.

### BRIEF DESCRIPTION OF THE DRAWINGS

FIG. 1 is a gas flow diagram of a gas supply section in an example of a radiative artificial respiration system with a carbon dioxide absorbent according to the present invention;

FIG. 2 is a gas flow diagram showing a low-flow anesthesia method according to an example of the present invention;

FIG. 3 is a gas flow diagram showing a low-flow anesthesia method according to a comparative example of the present invention;

FIG. 4 is a perspective view showing an example of a canister according to the present invention;

FIG. 5 is a vertical sectional view showing the example of the canister according to the present invention; and

FIG. 6 is a graph showing the non-load radiation characteristic of the canister of the present invention.

### DETAILED DESCRIPTION OF THE PREFERRED EMBODIMENT

With a closed circulatory artificial respiration system according to the present invention, the required water content is presumably maintained by heat radiation of a carbon dioxide absorbent; however, temperature variations in the circuit of the artificial respirator may result in the formation of condensation. If the carbon dioxide absorbent is cooled to 20° C., the concentration of decomposed compounds in the circuit presumably decreases, whereas the water content of the carbon dioxide absorbent may increase to affect the absorption and permeation of carbon dioxide. To reduce the variation in the temperature in the circuit and maintain the humidity of the carbon dioxide absorbent at an appropriate value, it is effective to maintain the temperature of the carbon dioxide absorbent between 20 and 40° C., and more preferably between 30 and 35° C.

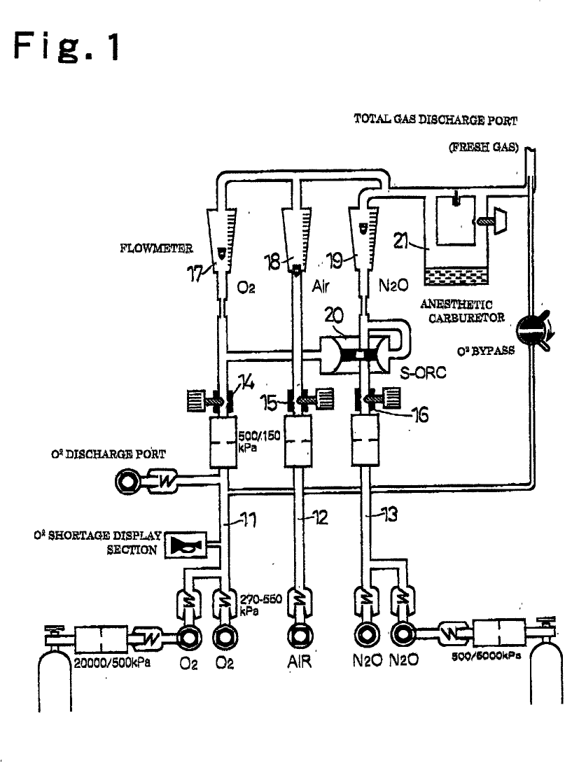

FIG. 1 shows an apparatus that forms a fresh gas and can supply oxygen, air, and nitrous oxide from the respective gas sources (20% or more of oxygen). When these gases pass collectively through an anesthetic carburetor, an anesthetic gas is supplied to these gases, so that the mixed gas is fed into an artificial respirator circuit as a fresh gas. Low-flow anesthesia contains 2 l/min. or less of fresh gas, while high-flow anesthesia contains more than 2 l/min. of fresh gas. The fresh gas is cold and dry.

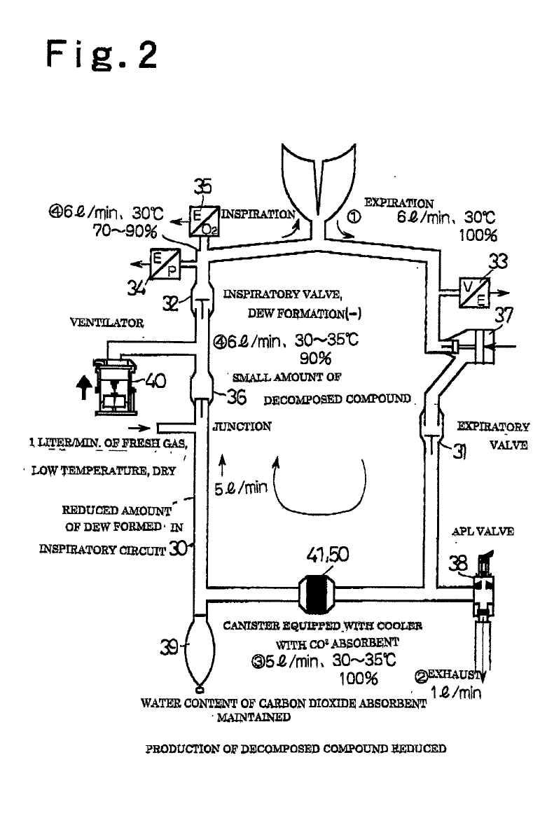

In view of the formation of condensation in the artificial respirator and adverse effects on the water content of the carbon dioxide absorbent, it is desirable to minimize variations in the temperature in the artificial respirator circuit (including the temperature in a canister). According to the present invention, the case of the canister can be formed of aluminum or copper, which has a high heat conductivity, or a material having an equivalent heat conductivity, so as to release internal heat, and a number of panels made of the same type of material can be installed inside the canister in a laminar manner so as to reduce variations in the temperature inside the canister. Furthermore, a Peltier element and a temperature controller are used to radiate heat from the carbon dioxide absorbent so as to maintain its temperature between 20 and 40° C. and more preferably between 30 and 35° C. This minimizes increases in the temperature of the carbon dioxide absorbent even in low-flow anesthesia to maintain the temperature in the artificial respirator circuit within the range specified above. This in turn reduces the production of toxic decomposed compounds and prevents condensation from being formed in an inspiratory circuit (FIG. 2).

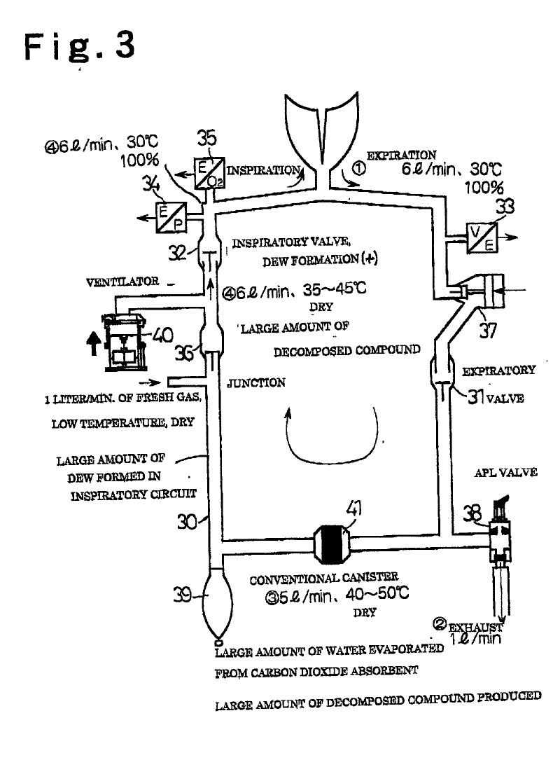

Examples and comparative examples of the present invention will be described below in detail with reference to the drawings; in these examples, the closed circulatory anesthetic circuits manufactured by Drager and called “Fabius” were used. In FIG. 1, reference numerals 11, 12, and 13 denote supply pipes for oxygen, air, and nitrous oxide, respectively; 14, 15, and 16 denote throttle vales provided in the respective supply pipes; 17, 18, and 19 denote flowmeters for the respective gases; 20 denotes an oxygen mixture ratio regulator; and 21 denotes a section that supplies a volatile anesthetic and which is shown as an anesthetic carburetor. Further, in FIGS. 2 and 3, reference numeral 30 denotes an artificial respirator circuit, shown as a circulation circuit; 31 denotes an expiratory valve, shown as a check valve; 32 denotes an inspiratory valve similar to the expiratory valve; 33 denotes a flowmeter; 34 denotes a pressure sensor; 35 denotes an oxygen sensor; 36 denotes a check valve at a junction; 37 denotes a PEEP/Pmax (positive end-expiratory pressure/maximum pressure) control valve; 38 denotes an APL (adjustable pressure limiting) valve; 39 denotes a pressurizing means, shown as a manual ventilation bag; 40 denotes a ventilator; and 41 denotes a canister.

### EXAMPLE 1

Low-flow Anesthesia Using the Cooler with the Carbon Dioxide Absorbent (Fresh Gas Flow is 1 l/min.; FIG. 2)

\mathring{1} Expiration: Contains oxygen, carbon dioxide, and an anesthetic gas. In-circuit flow is about 6 l/min. (if for an adult, single ventilation volume is 500 ml, the number of inspirations is 12, and ventilation minute volume is 6,000 ml/min.), temperature is 30° C., and humidity is 100%.

\mathring{2} Exhaust: Contains oxygen, carbon dioxide, and an anesthetic gas. In low-flow anesthesia (fresh gas flow: 1 l/min.), exhaust volume is about 1 l/min.

\mathring{3} Canister: The remaining gas of about 5 l/min. flows into the canister at a temperature of 30° C. and a humidity of 100%. Carbon dioxide reacts with the carbon dioxide absorbent to generate heat, but the canister has a high heat conductivity, thereby minimizing increases in the temperature of the carbon dioxide absorbent (30 to 35° C.). This reduces evaporation of water from the carbon dioxide absorbent to lessen the production of toxic decomposed compounds.

\mathring{4} Inflow and inspiration of fresh air: Fresh gas (flow: 1 l/min.) and 5 l/min. recycled gas join each other, and the joined gas is slightly cooled before about 6 l/min. gas is taken in by the patient at a temperature of about 30° C. and a humidity of 70 to 90% using a ventilator. The inspiration contains only a very small amount of decomposed compound. The difference in temperature between the carbon dioxide absorbent and the sucked gas is small, thus reducing the amount of condensation formed in the inspiratory circuit.

### COMPARATIVE EXAMPLE 1

### Low-flow Anesthesia Using a Conventional Canister (Fresh Gas Flow is 1 l/min.; FIG. 3)

\mathring{1} Expiration: Contains oxygen, carbon dioxide, and an anesthetic gas. In-circuit flow is about 6 l/min. (if single ventilation volume for an adult is 500 ml, the number of inspirations is 12, and minute ventilation volume is 6,000 ml/min.), temperature is 30° C., and humidity is 100%.

\mathring{2} Exhaust: Contains oxygen, carbon dioxide, and an anesthetic gas. In low-flow anesthesia (fresh gas flow: 1 l/min.), the exhaust volume is about 1 l/min.

\mathring{3} Canister: The remaining gas of about 5 l/min. flows into the canister at a temperature of 30° C. and a humidity of 100%. Carbon dioxide reacts with the carbon dioxide absorbent to generate heat, thereby locally increasing the temperature of the carbon dioxide absorbent to 40 to 50° C. or higher. A large amount of toxic decomposed compounds are produced, and a large amount of water is evaporated from the carbon dioxide absorbent.

\mathring{4} Inflow and inspiration of fresh air: Fresh gas (flow: 1 l/min.) and 5 l/min. recycled gas join each other, and the joined gas is cooled before about 6 l/min. gas is taken in by the patient at a temperature of about 30° C. and a humidity of 100% using a ventilator. The inspiration contains a large amount of toxic decomposed compounds. A large amount of water is evaporated from the carbon dioxide absorbent, which has become hot, and the temperature in the inspiratory circuit decreases rapidly to form condensation in the inspiratory circuit.

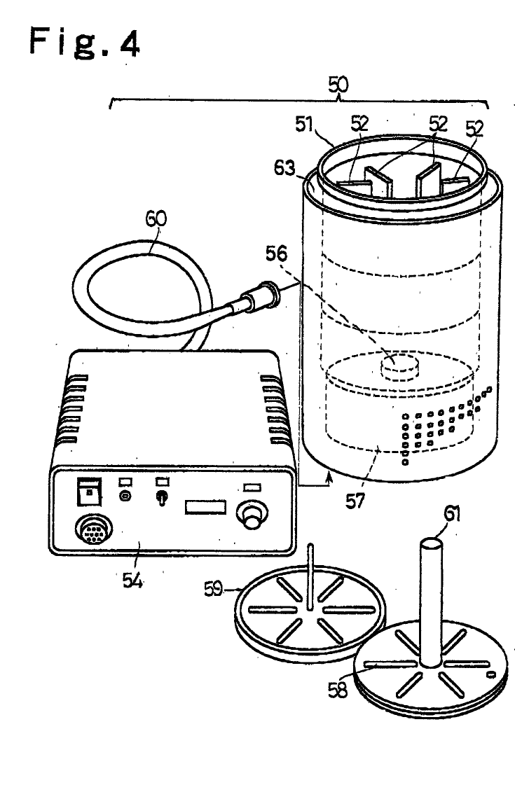

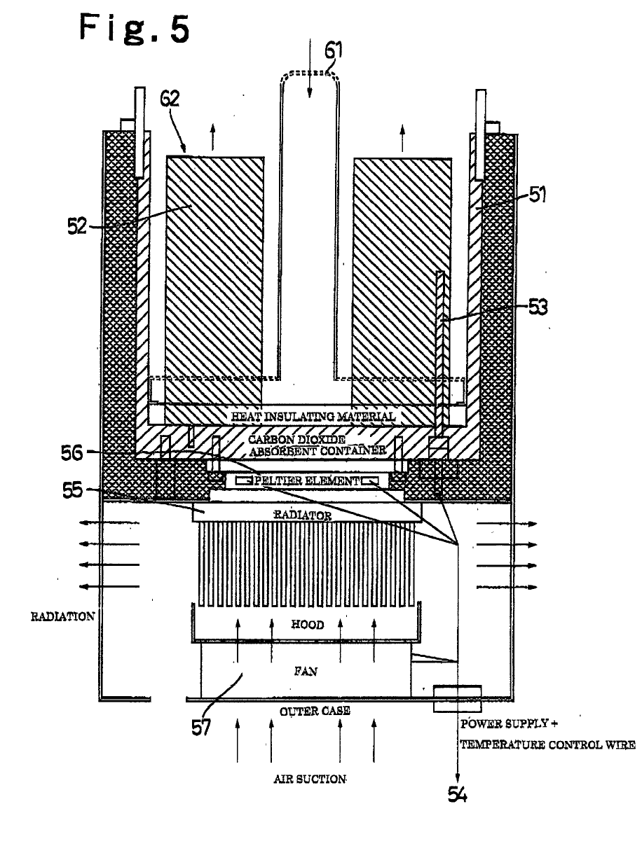

FIGS. 4 and 5 show an example of a canister connected to a radiator with a carbon dioxide absorbent according to the present invention to which the Peltier cooling method has been applied. In FIGS. 4 and 5, a canister 50 has a container case 51 made of aluminum, which has a high heat conductivity and which is plated with chromium in this example, and a plurality of radiator panels 52 disposed at uniform intervals and also made of chromium-plated aluminum. To reduce variations in the temperature inside the canister, the area of the radiator panels can be increased relative to the amount of carbon dioxide absorbent accommodated in the case. Furthermore, the apparatus is provided with at least one temperature sensor 53 for detecting the temperature inside the canister, a temperature controller 54 for maintaining the temperature of the carbon dioxide absorbent at an arbitrary value, and a radiator 55, a Peltier element 56, and a fan as a temperature-control means. Reference numeral 58 denotes a canister insert, 59 is a bottom insulating panel that prevents condensation from being formed in the canister, and 60 is a connection cable. Expiration flows in and out through an inflow port 61 and an outflow port 62 on the canister 50, respectively. Reference numeral 63 denotes a heat insulating section.

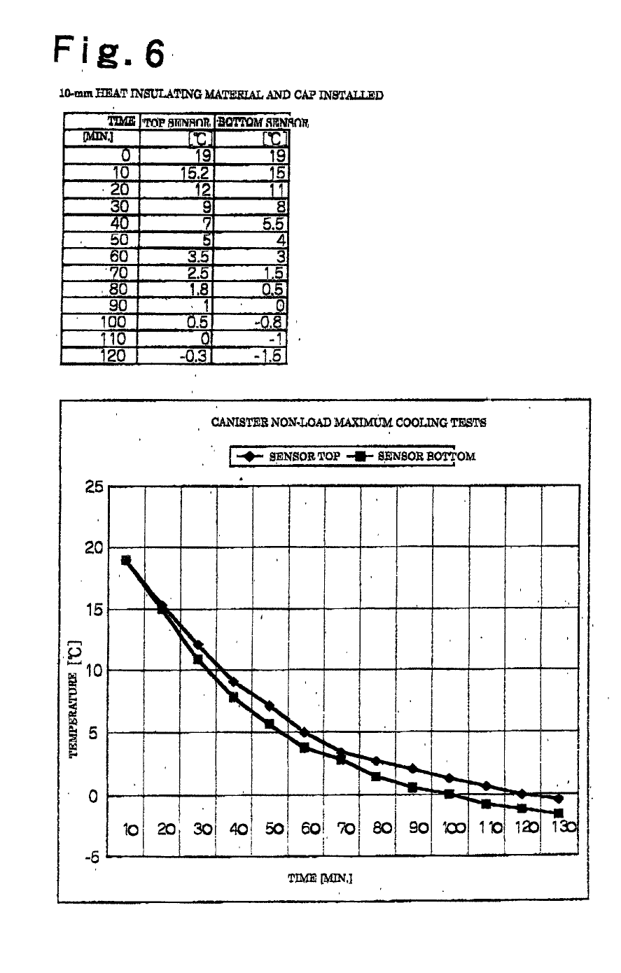

The canister 50 has the carbon dioxide absorbent set therein together with the canister insert 58 and the bottom insulating panel 59, and is applied to the artificial respirator circuit in FIG. 2. FIG. 6 is a graph showing numerical values and a graph obtained by conducting non-load radiation tests under the above conditions. The heat-radiating characteristic indicated in this figure clearly shows that the carbon dioxide absorbent could be maintained within the intended temperature range.

The present invention is constructed and operates as described above, so that even with the low-flow anesthesia method, increases in the temperature of the carbon dioxide absorbent can be minimized to reduce the amount of condensation formed in the inspiratory circuit, and thus the concentration of toxic decomposed compounds in the artificial respiration circuit is reduced as well. Therefore, the present invention has the effect of alleviating the above described problems with low-flow anesthesia, and also of reducing its environmental impact and medical costs, for example, by reducing the consumption of nitrous oxide, oxygen, and volatile anesthetics.

What is claimed is:

1. A radiative artificial respiration system with a carbon dioxide absorbent comprising a closed circulatory artificial respiration system that repeats a process of using a carbon dioxide absorbent to absorb carbon dioxide contained in expiration discharged from a patient in order to remove the carbon dioxide, then supplying oxygen and an anesthetic into the circuit in order to mix these gases with fresh gas, and subsequently allowing the patient to absorb the mixed gas as an inspiration, wherein when a low-flow anesthesia method is to be carried out, in which the total amount of the fresh air containing the oxygen and volatile anesthetic is 2 l/min. or less, the carbon dioxide absorbent, which generates heat when absorbing carbon dioxide, is allowed to efficiently radiate heat to minimize increases in the temperature of the carbon dioxide absorbent, thus reducing the amount of condensation formed in the anesthetic circuit, but without increasing the amount of decomposed compounds produced by the reaction between the volatile anesthetic and the carbon dioxide absorbent.

2. A radiative artificial respiration system with a carbon dioxide absorbent according to claim 1, wherein an air cooling method or a Peltier cooling method is added to radiate heat from the carbon dioxide absorbent.

3. A radiative artificial respiration system with a carbon dioxide absorbent comprising a closed circulatory artificial respiration system that repeats a process of using a carbon dioxide absorbent to absorb carbon dioxide contained in expiration discharged from a patient in order to remove the carbon dioxide, then supplying a fresh gas containing oxygen and an anesthetic into the circuit in order to allow it to combine with recycled gas, and subsequently allowing the patient to absorb fresh(mixed) gas as an inspiration, wherein when a low-flow anesthesia method is to be carried out, in which the total amount of the fresh air containing the oxygen and volatile anesthetic is 2 l/min. or less, the temperature of the carbon dioxide absorbent, which absorbs carbon dioxide, is maintained between 20 and 40° C. to suppress the reaction between the volatile anesthetic and the carbon dioxide absorbent.

4. A radiative artificial respiration system with a carbon dioxide absorbent according to claim 3, wherein the temperature of the carbon dioxide absorbent is maintained between 30 and 35° C.

5. A canister used for a radiative artificial respiration system with a carbon dioxide absorbent comprising a closed circulatory artificial respiration system that repeats a process of using a carbon dioxide absorbent to absorb carbon dioxide contained in expiration discharged from a patient in order to remove the carbon dioxide, then supplying a fresh gas containing oxygen and an anesthetic into the circuit in order to allow it to combine with recycled gas, and subsequently allowing the patient to absorb fresh(mixed) gas as an inspiration, wherein when a low-flow anesthesia method is to be carried out, in which the total amount of the fresh air containing the oxygen and volatile anesthetic is 2 l/min. or less, a case of the canister is formed of aluminum or copper, or a material having a heat conductivity equivalent to that of aluminum or copper, so as to radiate heat from the carbon dioxide absorbent, and radiator panels installed inside the case are made of the same type of material.
